(19) United States
(12) Patent Application Publication
(10) Pub. No.: US 2017/0052390 A1
(43) Pub. Date: Feb. 23, 2017

(54) ANTI-GLARE CORRECTION LENS

(71) Applicant: HSIAO-CHING TUNG, NEW TAIPEI CITY (TW)

(72) Inventor: HSIAO-CHING TUNG, NEW TAIPEI CITY (TW)

(21) Appl. No.: 14/929,420

(22) Filed: Nov. 2, 2015

(30) Foreign Application Priority Data
Aug. 21, 2015 (TW) 104127317

(51) Int. Cl. G02C 7/04; A61F 2/16

(52) U.S. Cl. CPC G02C 7/049; A61F 2/16; A61F 2002/1696

(57) ABSTRACT

An anti-glare correction lens has a central optical zone and a peripheral optical zone, which respectively define, with respect to the optical axis, a first focal point within a range of 2 degrees and a second focal point within a range of 2-10 degrees, and achieves a multi-focal correction function. Front curvatures or back curvatures are arranged to have curvatures that are varied radially outward from the central optical zone by each incrementing at least 2 diopters so as to define an aspheric configuration. The anti-glare layer absorbs internal reflection caused by the rear arc sections so as to prevent occurrence of glare.

### (a) TECHNICAL FIELD OF THE INVENTION

The present invention relates generally to an anti-glare correction lens that involves an aspheric lens design to achieve multiple focuses and correction of ametropia or refractive error and also shielding scattering light resulting from straight light, reflected or refracted light of the surroundings.

### (b) DESCRIPTION OF THE PRIOR ART

Presbyopia is a condition for which no entirely suitable permanent treatment has been developed. The most common solution is to wear spectacle glasses. The spectacle glasses used to correct the problem of presbyopia can comprise two pairs of single vision glasses, a single pair of multi-focal glasses having multiple focal points, or simply wearing contact lenses with a multi-focal design. Using two pairs of eyeglasses is surely an inconvenient solution. The multi-focal lenses may involve a translating vision lens, which requires a specific posture of head tilting down or gazing downward for reading. This is surely inconvenient. Alternatively, a simultaneous vision lens may be used for the multi-focal lenses, but it is also not satisfactory for severe presbyopia patients.

Another approach to treat ametropia is wearing rigid contact lenses having different refractive powers or diopters to gradually change the shape of corneas. Contact lenses of such purposes may continuously exert pressure to selected locations of a cornea to gradually force or mold a surface of the cornea into a desired shape. However, the rigid contact lens is generally not comfortable and this is hard to overcome. Some trials reshape the cornea by using an inverted (inside out) soft contact lens. The results of such use, however, have been proved unpredictable. The cost and durability of hybrid lenses, however, have been a concern.

Further, due to different refractive power on the lens surface of a contact lens, the direct or refracted light from the surroundings may impact the inner surface of the lens and generate internal reflection, which could be within the lens or in the eyeball based upon the location of light incidence. When the internal reflection enters and influences the pupil area, the light array may compromise the vision for its clarity and the field depth that is referred to as glare.

### SUMMARY OF THE INVENTION

The present invention aims to provide an anti-glare correction lens that comprises an aspheric surface design for multifocal correction of ametropia or refractive errors for better shielding of the scattering light resulting from straight, reflected or refracted light of the surroundings.

The primary object of the present invention is: a subject is first examined by a practitioner in order to determine the type and degree of ametropia, then select or design a soft contact lens having appropriate front and back surface curvatures to treat ametropia or correct the presbyopia with the multifocal designs, while also eliminating glare that can be induced by the aspheric lens.

To achieve the above purpose, present invention provides an optical lens comprises: a center optical zone in a central portion of the lens and a peripheral optical zone located adjacent to and radially outwardly from the center optical zone. A pressure control zone incorporated to and extends radially outward from the optical zone. An alignment zone incorporated to and extends radially outward from the pressure control zone. A plurality of curvatures and zones are formed on front surfaces of the optical zone, the pressure control zone, and the alignment zone that are on the rear surface. An anti-glare layer is formed in front of the pressure control zone or the alignment zone to absorb internal reflection light that is reflected into the pupil area from the rear surface of the lens. When a user wears the correction lens of the present invention, the curvature of either the front surface or the back surface of the lens in the peripheral optical zone is different from the curvature of the front surface or the back surface of the center optical zone by at least 2 diopters, to form two non-coincident foci in different location for multifocal correction. Furthermore, the front contour and the back contour of the pressure control zone and the alignment zone of the lens may convey the entire cornea contour and the eyelid force transmitted backward to conduct cornea molding or reshaping and correct ametropia. Further, the anti-glare layer is incorporated between the pressure control zone and the alignment zone to shield or absorb the light that is abnormally refracted or reflected to the pupil to prevent the wearer from the influence of the undesired light for a better field depth and vision clarity. It may also achieve a purpose of beautifying eyes by the colorful shape designs of the anti-glare layer.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings with identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a front view of the present invention.

FIG. 2 is a cross-sectional view taken along line A-A.

FIG. 3 is a schematic view illustrating the use of the present invention.

FIG. 4 is a schematic view illustrating multiple focal points of the present invention.

FIG. 5 is another schematic view illustrating the multiple focal points of the present invention.

FIG. 6 is a schematic view illustrating an example of vision correction according to the present invention.

FIG. 7 is a schematic view illustrating an example of glare resistance according to the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

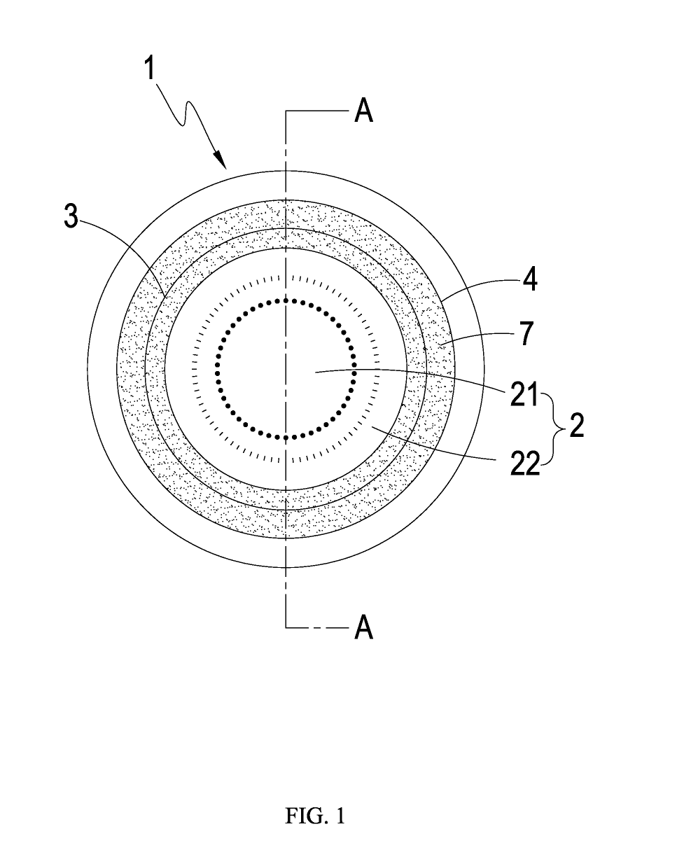

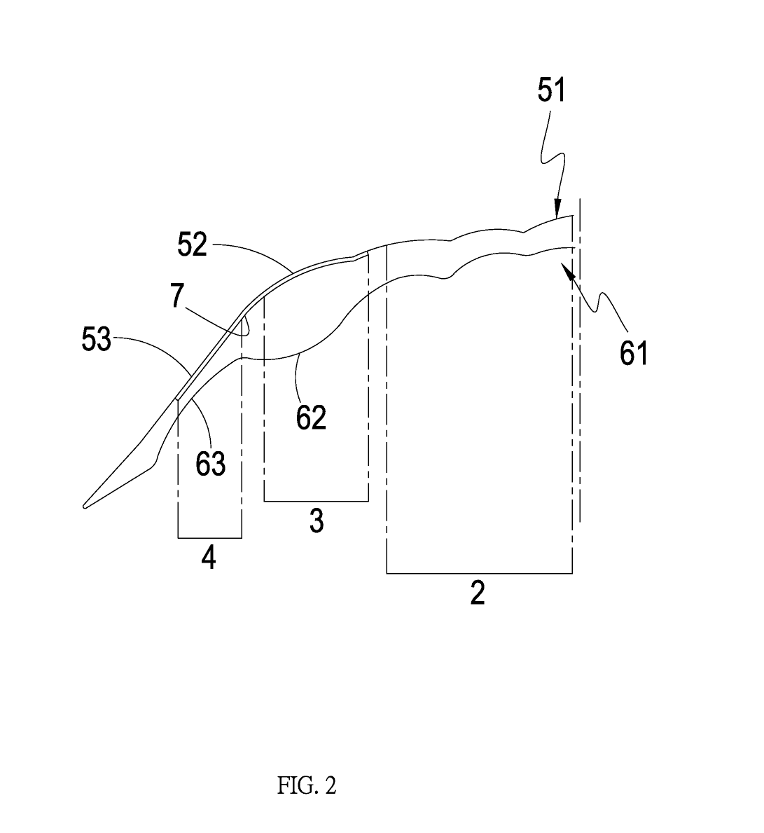

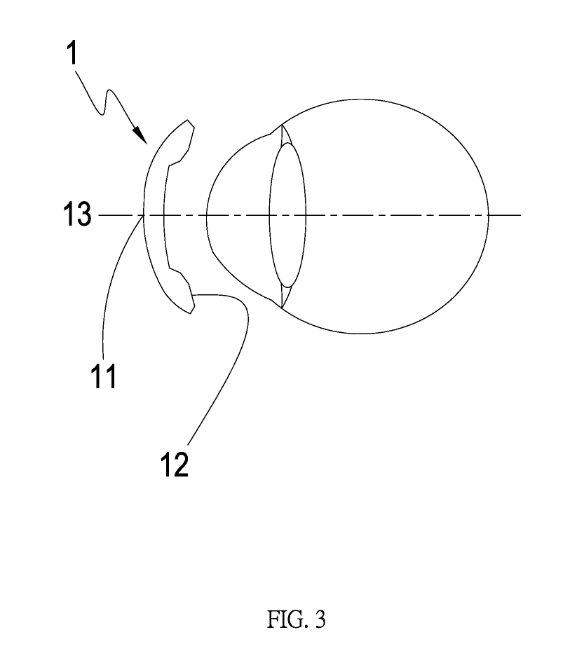

Referring to FIGS. 1-7, the drawings clearly show the present invention provides a correction lens 1, which is a correction lens that has a front surface 11, a back or rear surface 12, and an optical axis 13. The correction lens 1 comprises:

an optical zone 2, the optical zone 2 comprising a central optical zone 21 located at a center of the correction lens 1 and a peripheral optical zone 22 adjacent to and extending radially outwards from the central optical zone 21, wherein the center optical zone 21 focuses light that enters the front surface 11 of the lens in a direction substantially parallel to the optical axis 13 of the lens to create a first focal point A within 2.5° of the optical axis 13, while the peripheral optical zone 22 focuses light in a direction intersecting the direction of the optical axis 13 to create a second, non-overlapping focal point B at between 2° and 10° with respect to the optical axis 13;

a pressure control zone 3 extending radially outward from the optical zone 2;

an alignment zone 4 extending radially outward from the pressure control zone 3;

a plurality of front curvatures 5 and back curvatures 6 formed respectively on the front surface 11 and the back surface 12 to define the optical zone 2, the pressure control zone 3, and the alignment zone 4, wherein the front curvatures 5 define a front optical curve 51, a front pressure control curve 52, and a front alignment curve 53 respectively for individual zones and the back curvatures 6 comprise a base curve 61, a pressure control back curve 62, and an alignment back curve 63 respectively for each individual zone, and wherein the curvature of either the front curvatures 5 or the back curvatures 6 of the lens having the optical zone 21 that becomes progressively steeper radially outwardly with an increment of at least 2 dipoters so that the inner radial curve and the outer radial zone are different from each by 2-10 diopters to thus define an aspheric contour; and

an anti-glare layer 7 arranged on the pressure control zone 3 or the alignment zone 4 to absorb or shield internal reflection light that is reflected by the back curvatures 6 toward the pupil area, wherein the anti-glare layer 7 is arranged to extend, radially outward, from a site that is 1.5-7.5 mm distant from the central point of the correction lens 1 and having a thickness of around 1 μm-1 mm, with an annular or circumferential ring like configuration, wherein the annular ring having a width of 0.1-6 mm.

To manufacture the lens according to the present invention, the hardness of the lens is preferably set to be substantially corresponding to that of the conventional soft contact lenses. Thus, the material can be made from the group materials of Lotrafilcon A, Balafilcon A, Lotrafilcon B, Comfilcon A, pHEMA (polyhydroxyethylmethacrylate), Omafilcon A, and Galyfilcon A. Lenses having a hardness which is greater than that of such soft lenses can also be used, as long as the rear surface 12 of the lens generally assumes the contour of a corneal surface when placed onto a cornea.

The anti-glare layer 7 can be made of a material selected from water-based or oil-based colorants (such as ink), carbon black, organic and inorganic dyestuffs, pigments, light-shielding agents or light reflecting agents (such as titanium oxide), aluminum oxide pearl powder/shell powder, photochromic agents, or thermochromic agents, or a mixture or polymer of the materials, and is manufactured to provide a ring-like pattern having an absorbing or shielding function. The pattern may be in the form of or involves a continuous or non-continuous pattern of mesh, dots, strips, blocks, squares, circles, triangles, heart-shapes, star-shapes, or polygons.

The contact lens of the present invention can be manufactured with the conventional ways that are known in the industry, such as lathing, spin casting, or cast molding, or soft cast molding, such as fully hydrated or partially hydrated, using a glass mold for cast molding, where the size of a dry lens can be determined according to the expansion factor of the material to be used. The method used to make the anti-glare layer 7 can be a transfer-printing method, wherein a thin film on which a ring is first printed on and then transferred to an upper layer, a lower layer, or a middle layer of the contact lens, or a translation-printing method, where ink is first applied to form a pattern on a mold for printing a color ring on an upper layer, a lower layer, or a middle layer of a contact lens, or a spraying method, where ink jetting is applied to form a color ring on an upper layer, a lower layer, or a middle layer of a contact lens.

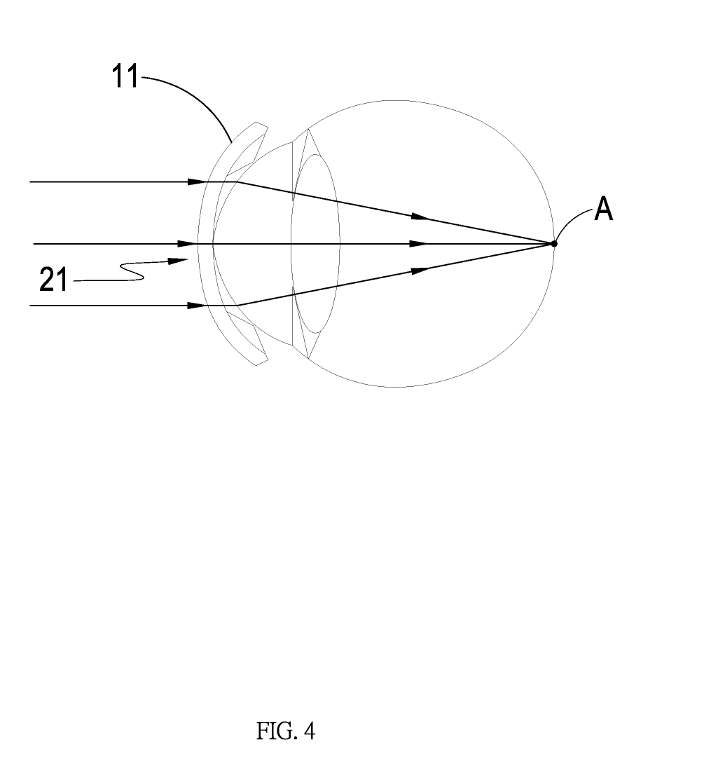

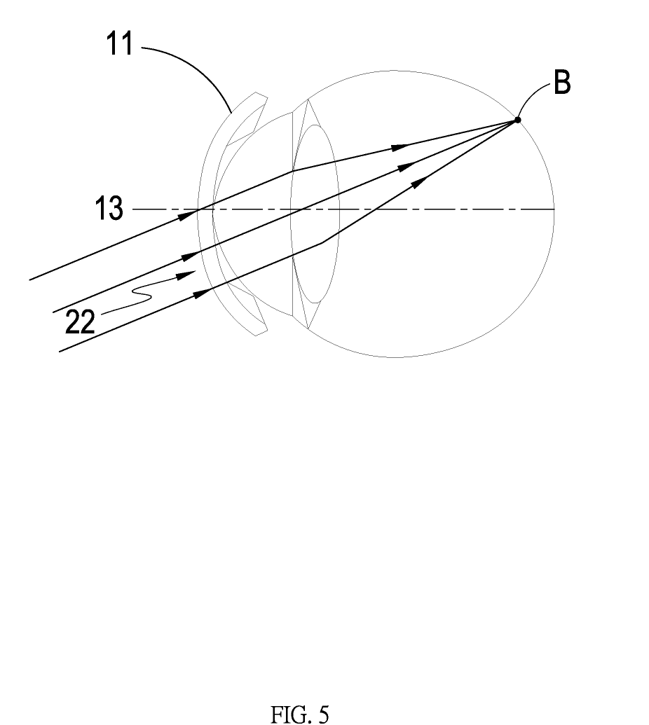

As shown in FIGS. 4 and 5, in practical use, an optical device of a contact lens or an intraocular lens provides a central optical zone 21, which has a refractive power for far vision correction subtending a visual angle of about 4-5 degrees corresponding to the 1.5 mm fovea of the macula located behind the plane of a human cornea for a distance of around 22.6 mm. Further provided is a near optical zone 22 adjacent to and radially outward from the central optical zone 21 of the device, having a shorter focal length or higher ADD to provide a near image that is much clearer than that formed by the central optical zone 21 and the difference is significant enough to trigger the off-axis parafovea and/or perifovea areas for PVS (preferential visual span) reading. The peripheral optical zone 22 of the optical device subtends a visual angle that is greater than the 4-5 degrees visual angle of the central fovea of macula but within 18-20 degrees respect to the visual center (or 9-10 degrees to each side of the optical axis 13 or visual axis), corresponding to the parafovea area (maximum 10 degrees) and perifovea area (maximum 20 degrees) for the visual span for reading. The contrast between far vision and near vision of the optical zone 2 is sufficient for human brain to distinguish images (texts) from the near vision optical zone 2 and recognized by the parafovea and perifovea, while ignoring coaxial burring images (texts) created by the far vision of the optical zone 2 recognized by the center of the central fovea. The clarity contrast between the far vision optical zone 21 and near vision optical zone 22 for the different focal lengths has to be significant enough for the brain to automatically select the clearer off-axis parafovea and/or perifovea images formed in peripheral optical zone 22 for perceptual comprehension, and neglect the on-axis blurrier central fovea images formed by the central optical zone 21.

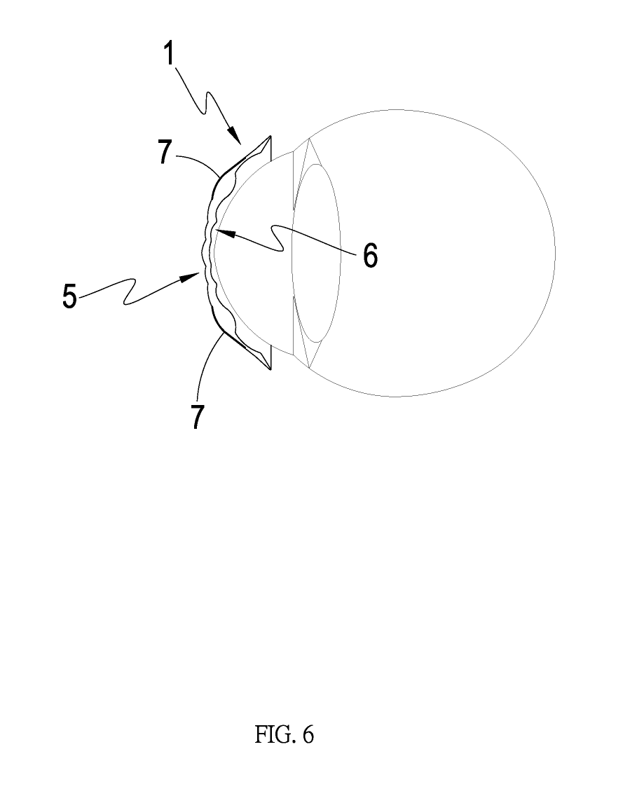

As shown in FIG. 6, the soft contact lens of the present invention is flexible and whose surface generally assumes the contour of a corneal surface when placed onto a cornea, particularly in a contact lens with thinner central thickness. A soft spherical contact lens does not form tear lens underneath the lens. Thus, the original cornea contour, such as the curvature and astigmatism, all will be transferred to the rear surface 12 of the soft contact lens and then further transferred to the front surface 11 of the soft contact lens 1. While, a rigid spherical contact lens may neutralize most corneal astigmatism of the ametropia and does not transfer to the front surface 11 (the power surface) of the contact lens. Thus, corneal reshaping by rigid contact lenses, including but not limited to the hydraulic massage and compression forces exerted by the optical zone 2 and alignment zone 4 of a rigid contact lens, is accomplished differently than in the present method for ortho-k using a soft contact lens 1.

While a soft contact lens 1 transfers the contour of the cornea to the back surface 12, then to the front surface 11 of the contact lens 1, force can also be applied in the opposite direction, i.e. from the eyelid to the front surface 11 of the soft contact lens 1 and then to the back surface 12 of the contact lens 1, and such force can ultimately be applied to the cornea. Hence, the front surface 11 and the back surface 12 of the soft contact lens 1, covering the same geometric site of the cornea, can be considered to function as a unit, such that any curvature or thickness changes made on either side will be transferred and reflected onto the corresponding area of the cornea for ortho-k or corneal reshaping.

A rigid ortho-k lens transfers the lid pressure to the surface of a cornea by consecutive contact or non-contact zones, via the rigidity of the material itself, to exert positive or negative pressure on areas of cornea to alter the corneal shapes accordingly. As aforementioned, current soft contact lenses are pliable and conform to the corneal surface, from center to periphery, via which the lid pressure is uniformly transferred to the entire cornea, and no alternate “positive and negative” forces are generated by such lenses for conducting ortho-k or corneal reshaping. A soft contact lens 1 can, however, generate relatively positive or negative forces by creating thickness differences in the soft contact lens material instead of curvature differences, to simulate the relative pressure of a rigid ortho-k contact lens for each zone of the lens. The relative thicknesses and/or curvatures of different zones of the soft contact lens will transfer lid pressure, in a form of relatively positive or negative forces, backward to the front surface of the cornea for corneal reshaping. A thinner zone applies relatively “negative force,” i.e. less force than a thicker zone, while a thicker zone exerts “positive force,” which is similar to what a rigid ortho-k contact lens can do with steeper or flatter zones on the back surface 12 of a rigid contact lens, to reshape a cornea.

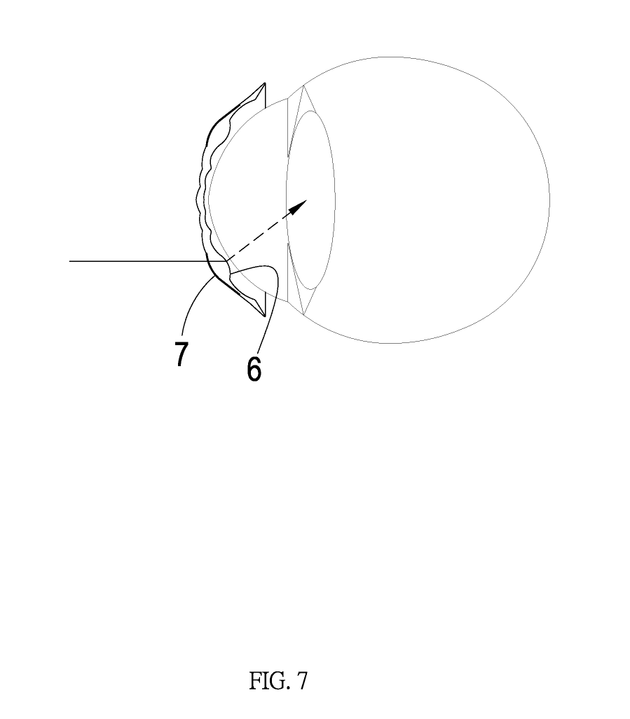

As shown in FIG. 7, the front curvatures 5 and the back curvatures 6 of the present invention, particularly the portions thereof associated with the pressure control zone 3 and the alignment zone 4, may, at the moment when surrounding light enter the interior of the lens as straight light or refracted light, generate internal reflection at the site of the back curvature 6 and it is possible that the light will be reflected to the pupil. This reflected light emerging from a resource outside of the visual axis would lead to blurry, fluffy or even ghost images that should not appear at the edges of the visual field, which is referred to as glare. Thus, with the anti-glare layer 7 arranged on the pressure control zone 3, the alignment zone 4, or between the pressure control zone 3 and the alignment zone 4, or on both the pressure control zone 3 and the alignment 4, the anti-glare layer 7 may shield or absorb the scattering light emerging from straight or refracted surrounding light; or the scatting light coming from internal reflection of the lens (phantom line arrow of the drawing indicating internal reflection light that has been absorbed or shielded), thereby achieving enhancement of the field of view with better contrast, and concentrating vision for less blurring of the vision, and may further achieve cosmetic or beautifying purposes by shielding and changing the iris color.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. An anti-glare correction lens, having a front surface, a rear surface, and an optical axis, the correction lens comprising:
a center optical zone in a central portion of the lens, wherein the center optical zone focuses light that enters the front surface of the lens in a direction substantially parallel to the optical axis to create a first focal point within 2.5° of the optical axis;
a peripheral optical zone located adjacent and radially outwardly from the center optical zone, wherein the peripheral optical zone focuses light that enters the front surface of the lens in a direction not parallel to the optical axis to create a second focal point at between 2° and 10° with respect to the optical axis;
a pressure control zone adjacent to and extending radially outward from the peripheral optical zone;
an alignment zone adjacent to and extending radially outward from the pressure control zone;
wherein the curvature of either the front surface or the back surface of the lens in the peripheral optical zone is steeper than the curvature of the front surface or the back surface of the lens in the center optical zone, respectively, by at least 2 to 10 diopters, and
an anti-glare layer arranged on the pressure control zone or the alignment zone to absorb or shield internal reflection light.

2. The anti-glare correction lens according to claim 1, wherein the anti-glare layer is arranged to extend radially outward, from a site having a distance from the central point of the correction lens by 1.5-7.5 mm.

3. The anti-glare correction lens according to claim 1, wherein the anti-glare layer has a circumferential ring like configuration, which has a width that is in a range of 0.1-6 mm.

4. The anti-glare correction lens according to claim 1, wherein the anti-glare layer has a thickness of 1 μm-1 mm.

5. The anti-glare correction lens according to claim 4, wherein the anti-glare layer is formed, by means of transfer printing, translation printing, or spraying, on the front surface, the rear surface, or a location between the front surface and the rear surface.

6. The anti-glare correction lens according to claim 1, wherein the correction lens is in the form of one of an intraocular lens, a soft contact lens, a rigid contact lens, and a soft-rigid composite contact lens.

7. The anti-glare correction lens according to claim 1, wherein the anti-glare layer has one of a single color, multiple colors, a mixed color, gradient color, photochromic color, and thermochromic color.

8. The anti-glare correction lens according to claim 1, wherein the correction lens has at least an axial thickness, the axial thickness being a distance measured, at a predetermined point on the correction lens, from the front surface to the rear surface in a direction parallel to the optical axis.

9. The anti-glare correction lens according to claim 8, wherein the pressure control zone has a minimum axial thickness that is less than a minimum axial thickness of the optical zone and a minimum axial thickness of the alignment zone, and the pressure control zone has a maximum axial thickness that is greater than a maximum axial thickness of the optical zone and a maximum axial thickness of the alignment zone.
